(19) United States
(12) Patent Application Publication
(10) Pub. No.: US 2015/0099965 A1
(43) Pub. Date: Apr. 9, 2015

(54) SYSTEM FOR HIGH RESOLUTION FAST ACQUISITION MAGNETIC RESONANCE IMAGING USING A CATHETER-MOUNTED COIL

(71) Applicant: UNIVERSITY OF UTAH RESEARCH FOUNDATION, Salt Lake City, UT (US)

(72) Inventors: Nelly A. Volland, Florence, SC (US); J. Rock Hadley, Centerville, UT (US); Dennis Lee Parker, Centerville, UT (US); Evgueni G. Kholmovski, Salt Lake City, UT (US); Robb Phillip Merrill, North Salt Lake, UT (US); Nassir F. Marrouche, Park City, UT (US)

(21) Appl. No.: 14/508,070

(22) Filed: Oct. 7, 2014

Related U.S. Application Data
(60) Provisional application No. 61/887,611, filed on Oct. 7, 2013.

(51) Int. Cl. G01R 33/28; A61M 25/00; A61M 25/10; G01R 33/48; A61B 5/00

(52) U.S. Cl. CPC G01R 33/287; G01R 33/48; A61B 5/6858; A61B 5/6853; A61M 25/1011; A61M 25/0074; A61M 25/005; A61B 5/055
USPC 600/423

(57) ABSTRACT

A catheter-mounted, expandable or set in position, coil for magnetic resonance imaging. The coil having a catheter sheath including an elongated tube with a central axis, the catheter sheath having an opening at an end thereof; an expandable coil including a conductive material connected to an expansion mechanism which, when deployed, maintains the expandable receive coil shape; and a cable running through the catheter sheath, the cable being electrically connected to the coil inductive loop.

### CROSS-REFERENCE TO RELATED APPLICATIONS

This application claims priority to co-pending U.S. Provisional Patent Application No. 61/887,611 filed on Oct. 7, 2013, the entire content of which is incorporated herein by reference.

### BACKGROUND

The present invention relates to high-resolution, fast acquisition magnetic resonance (MR) imaging using a catheter-mounted radiofrequency (RF) coil.

In certain catheter-based interventional applications, obtaining high sensitivity images of a localized treatment volume may be more important than obtaining low sensitivity images of the full anatomy. Even though 3D MRI measurement and monitoring capabilities have been increasingly used in such procedures, defining tissue microstructure using diffusion tensor imaging (DTI), measuring temperature during thermal ablation treatment, determining local tissue perfusion, or performing MR spectroscopy (MRS) for detecting metabolites are some of the applications requiring sensitivity enhancements in order to be clinically viable. However, currently available external RF coils limit the image acquisition to large field-of-view (FOV) covering the full anatomy to prevent aliasing. These settings are associated with low signal-to-noise (SNR) in the region of interest (ROI) and slow speed image acquisition often inadequate for obtaining high sensitivity images of a localized treatment volume.

### SUMMARY

Accordingly, disclosed herein is the development of a system for limited FOV high resolution, high SNR MR measurements, which in some embodiments is a catheter-mounted expandable loop (CAMEL) RF coil for intracardiac MRI and in other embodiments is a catheter-mounted RF coil in a set position for applications such as renal artery MRI.

One or more of soft tissue visualization, interventional catheter navigation and localization, and procedure outcomes will benefit from the disclosed system.

In one embodiment, the invention provides an expandable catheter-mounted RF coil for magnetic resonance imaging. The coil includes a catheter sheath having an elongated tube with a central axis, the catheter sheath having an opening at an end thereof; an expandable coil including a conductive material connected to an expansion mechanism which, when deployed, maintains the expandable coil in a defined shape; and a cable running through the catheter sheath, the cable being electrically connected to the expandable coil.

In another embodiment the invention provides a method for performing magnetic resonance imaging. The method includes the steps of providing a catheter sheath including an elongated tube with a central axis, the catheter sheath having an opening at an end thereof; disposing a cable within the catheter sheath; electrically connecting an expandable coil to the cable, the expandable coil comprising a conductive material connected to an expansion mechanism; deploying the expansion mechanism to maintain the expandable coil shape; and placing the expandable coil within a magnetic field to obtain data.

In still another embodiment the invention provides a method for performing magnetic resonance imaging. The method includes the steps of providing a catheter sheath having an elongated tube with a central axis, the catheter sheath having an opening at an end thereof; disposing a cable within the catheter sheath; electrically connecting a coil to the cable, the coil including a conductive material; maintaining the coil shape; and locating the catheter sheath with the coil within a magnetic field to obtain data.

Other aspects of the invention will become apparent by consideration of the detailed description and accompanying drawings.

### BRIEF DESCRIPTION OF THE DRAWINGS

FIG. 1 shows an umbrella-type CAMEL coil including diagrams of the coil circular loop (a) in the expanded position and (b) the folded position; photographs of an embodiment of an umbrella-type coil (c) in the expanded position, (d) the half-folded position, and (e) the folded position in the catheter sheath.

FIG. 2 shows a balloon-type CAMEL coil including diagrams of the coil circular loop (a) in the expanded position, (b) cross-sectional view of the expanded position sectioned parallel to the long axis, (c) the folded position, and (d) cross-sectional view of the folded position sectioned perpendicular to the long axis; photographs of an embodiment of a balloon-type coil (e) in the expanded position in a front perspective view, (f) in the expanded position in a side view, and (g) in the folded position in a side view.

FIG. 3A shows a circuit diagram for a catheter-mounted coil. In one embodiment a 24 cm long micro-coaxial cable is used to connect the RF coil to the receiver circuit board and a 24 cm long coaxial cable is used to connect the receiver circuit to the preamplifier board, which may include a cable trap to minimize common-mode currents on the cable, a phase shifter to maintain λ/2 phase shift between the coil, and a low input impedance preamplifier.

FIG. 3B shows a photo montage of one type of catheter-mounted coil (bottom) adjacent to a circuit diagram (top) as in FIG. 3A, indicating which portions are disposable and inserted into the subject, which are non-disposable and outside of the subject, and which are scanner-specific electronics components.

FIG. 4A shows MR magnitude and phase images and intensity profiles for the umbrella-type CAMEL coil in a homogeneous phantom. Zoomed-in images: magnitude images are shown in the (a) coronal; (b) axial; and (c) sagittal view; phase images are shown in the (d) coronal; (e) axial and (f) sagittal views; intensity profiles are shown: (g) for the magnitude images; and (h) for the phase images. The original FOV for these images was 192×192 mm². In FIGS. 4A, 4B, and 4C, the horizontal (circle) and vertical (square) lines in panel (a) and the vertical lines in panels (b) (cross) and (c) (triangle) are graphed in panel (g) using the symbols as indicated; similarly, the horizontal (circle) and vertical (square) lines in panel (d) and the vertical lines in panels (e) (cross) and (f) (triangle) are graphed in panel (g) using the symbols as indicated.

FIG. 4B shows MR magnitude and phase images and intensity profiles for the balloon-type CAMEL coil in a homogeneous phantom. Zoomed-in images: magnitude images are shown in the (a) coronal; (b) axial; and (c) sagittal view; phase images in the (d) coronal; (e) axial and (f) sagittal views; Intensity profiles: (g) for the magnitude images; and (h) for the phase images. The original FOV for these images was 192×192 mm².

FIG. 4C shows MR magnitude and phase images and intensity profiles for the local coil in a homogeneous phantom. Zoomed-in images: magnitude images are shown in the (a) coronal; (b) axial; and (c) sagittal view; phase images in the (d) coronal; (e) axial and (f) sagittal views; Intensity profiles: (g) for the magnitude images; and (h) for the phase images. The original FOV for these images was 192×192 mm².

FIG. 5: High resolution images of a kiwi fruit phantom acquired with the umbrella-type coil. Zoomed-in (a) Magnitude image (b) phase image. The original FOV for these images was 96×96 mm².

FIG. 6A shows a catheter mounted circular microsolenoid coil. Panel (a) shows a side perspective view schematic of the coil at the distal end of the catheter sheath; panel (b) shows a cross-sectional perspective view of the catheter of panel (a) to show the electrical connections inside the sheath between the coil and the coaxial cable; panel (c) shows a perspective view of the distal end of the catheter sheath including the microsolenoid coil. The outer sheath is used for coil protection and isolation from body fluids. The inner sheath allows the RF ablation catheter to reach the tissue to be treated.

FIG. 6B shows a catheter-mounted curved circular microsolenoid coil. Panel (a) shows a perspective view schematic of the coil at the distal end of the catheter sheath; panel (b) shows a cross-sectional perspective view of the catheter in panel (a) to show the electrical connections inside the sheath between the coil and the coaxial cable; panel (c) shows a perspective view of the distal end of the catheter sheath including the microsolenoid coil. The curved shaped of the microsolenoid improves the sensitivity volume coverage in front of the coil where the ablation catheter is placed to create lesions in the tissue.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

To overcome sensitivity limitations, the disclosed system uses an RF coil positioned within the cardiac chambers. While intracavity coils have been developed, few are catheter-mounted expandable coils and even fewer can be used within the cardiac chambers. Although a few intracavity coils are commercially available, none are expandable nor are they suited for intracardiac applications. Recent advances in intravascular coils have focused mainly on catheter-mounted coils to image the blood vessel walls and not larger cavities. Possible designs include microsolenoid, loopless, and active stent designs. However, a conductive loop design allows the best detection profile for imaging the myocardium wall. To make the coil insertion realizable, the coil loop must be integrated with and deployed from a catheter sheath to enable insertion into the blood vessels before being expanded in the heart.

Disclosed herein is the development of a catheter-mounted expandable loop (CAMEL) coil for intracardiac MRI, including embodiments with various expansion mechanisms, materials, coatings, and designs for obtaining mechanical stability and reproducibility. The results demonstrate the use of the disclosed system for performing limited FOV, high resolution, high sensitivity MR measurements within tissues such as the myocardium prior to, during, and after catheter-based cardiac interventional procedures.

CAMEL Coil Construction

Several mechanisms were employed for catheter-mounted coil expansion, taking into account constraints such as a need for the coil to withstand several cycles of unfolding and folding that could occur for a given coil during one procedure (e.g. from 5-30 cycles). Ease of use by clinicians and the ability to be quickly retrieved were also important factors. Furthermore, prototypes had to be constructed so as to fit within a 12Fr (12 French=4 mm diameter) catheter sheath when the coil was in the folded position, since this is the largest diameter sheath to be placed in the test animal (dogs and pigs) for vascular access.

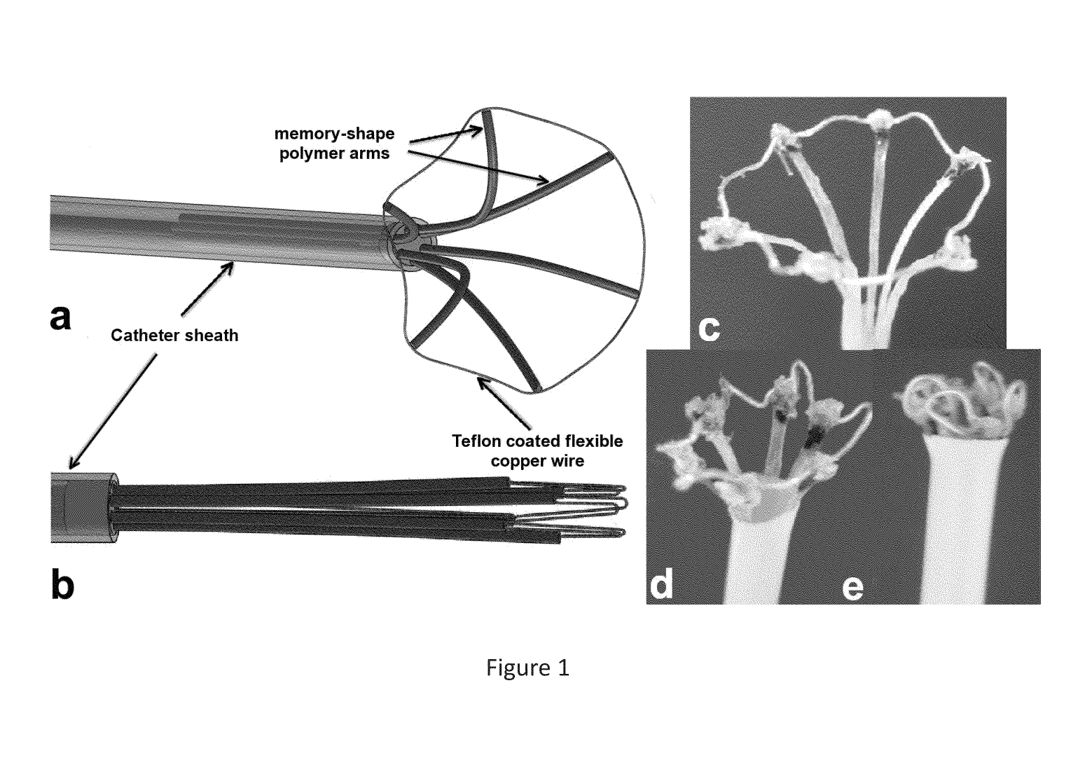

One embodiment of the CAMEL coil is referred to as an umbrella coil (FIGS. 1a-1e). In this embodiment the coil circular loop is anchored at the ends of six equally spaced thin solid elastic filaments, each of which was pre-formed into an arc (FIG. 1a). In various embodiments, the filaments may include ultra-high-molecular-weight-polyethylene or aramid materials, which have high tolerance for flexing and are not brittle or hard. These filaments allow for consistent folding and expansion of the 2-cm diameter loop of Teflon coated flexible (foldable) 38 AWG braided copper wire. To deploy the coil loop, the support filaments are pushed out of the catheter sheath until the loop is fully extended.

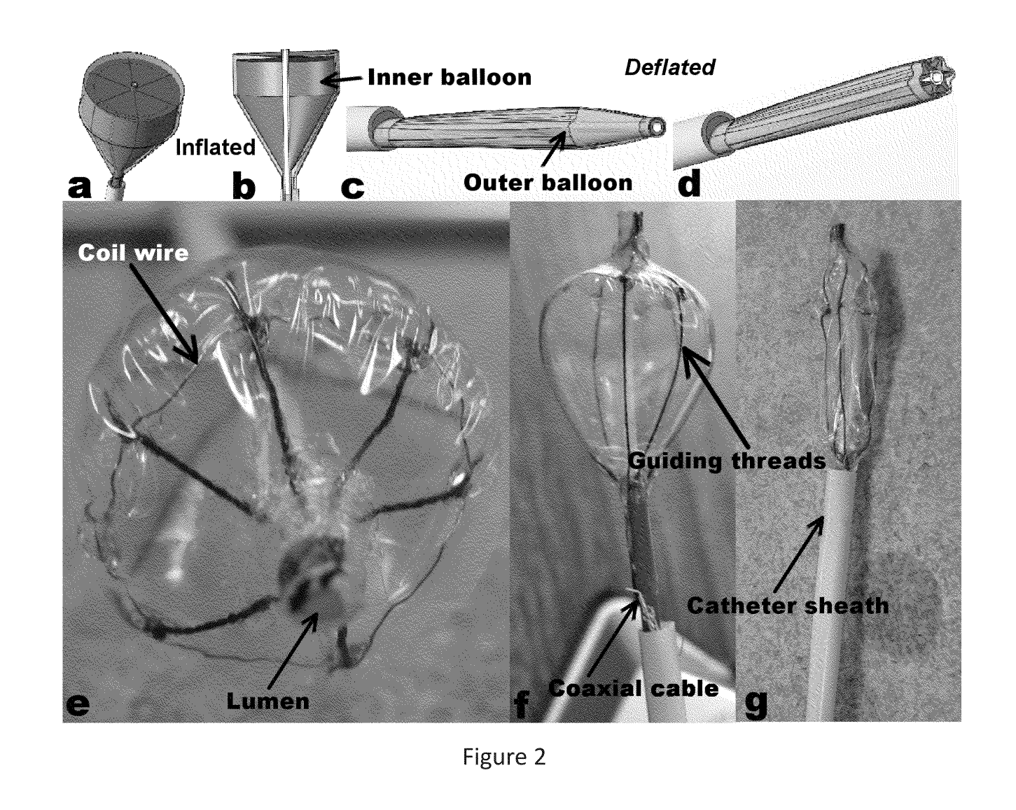

Another embodiment of the CAMEL coil is referred to as a balloon coil (FIGS. 2a-2g). In this embodiment the coil circular loop is anchored onto a balloon catheter (FIG. 2e). In this embodiment a 38 AWG braided copper wire loop is placed between an inner balloon with a series of longitudinally-placed threads to keep the coil in the correct position and an outer balloon of the balloon catheter. In general, the threads are connected to the coil at regular intervals and the thread and/or coil are attached at discrete locations around the perimeter of the balloon, for example using glue or other adhesive. In so doing, the coil and balloon are both permitted to fold more or less independently of one another when the balloon is retracted, which helps the balloon coil fold to a sufficiently small size to fit inside the catheter sheath. Anywhere from 2-10, or 4-8, or 6 threads may be used to secure the coil around the perimeter of the balloon. In various embodiments, other methods may be used to attach the coil to the balloon; for example, one or a series of channels may be associated with the balloon(s) (e.g. attached to or molded into the balloon(s)) into which the coil is threaded. In general, the method of attachment allows the coil to expand and contract with the balloon(s) such that in the expanded state the coil assumes a predictable and consistent configuration.

In various balloon embodiments, either a gas such as air or a liquid such as saline may be used to inflate the inner balloon and expand the coil; results of measurements using these two inflation mechanisms are compared. Since the use of saline within the inner balloon is expected to give a bright signal in MR and provide no contrast with the saline phantom, MnCl— doped saline was used to inflate the balloon during initial tests. The interaction between the manganese and the hydrogen atom of the water reduces the water hydrogen MR signal so no signal appears on the images from inside the balloon. Accordingly, in certain embodiments 5 mMol of MnCl₂ powder is added to the saline solution to null its MR signal inside the inner balloon. In other embodiments, liquids for expanding the balloon include various biocompatible, susceptibility-matched, and hydrogen signal-limited liquids such as Fluorinert (FC-43). Fluorinert does not include hydrogen, which would show on MR images. To ensure a complete and reproducible expansion of the coil, the six threads were equally spaced around the coil loop and anchored at the bottom and the top of the balloon, referred as to the inner balloon. An outer balloon was then placed over the inner balloon and the coil loop to protect the coil from contact with fluids.

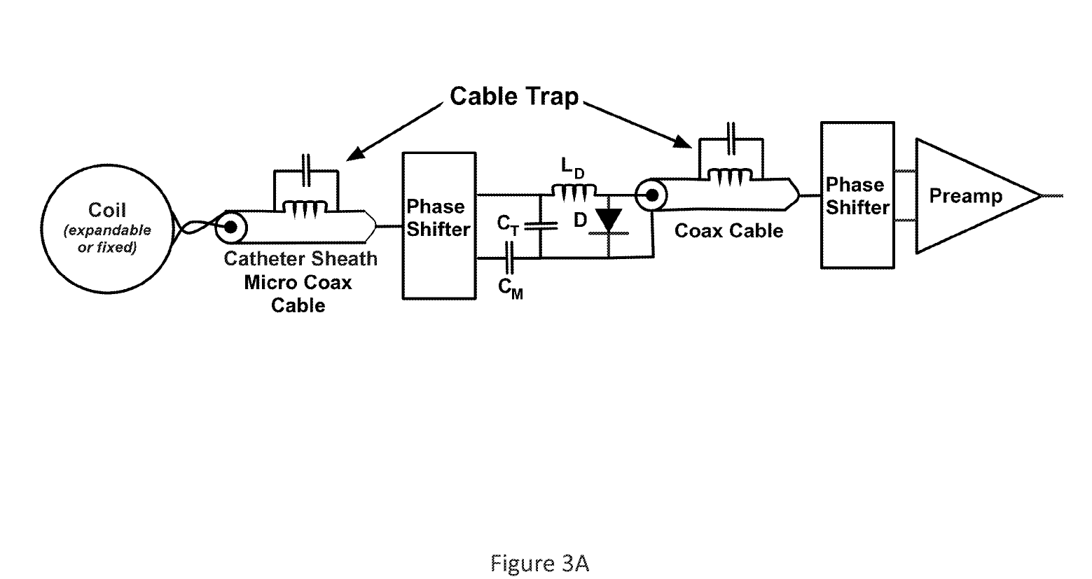

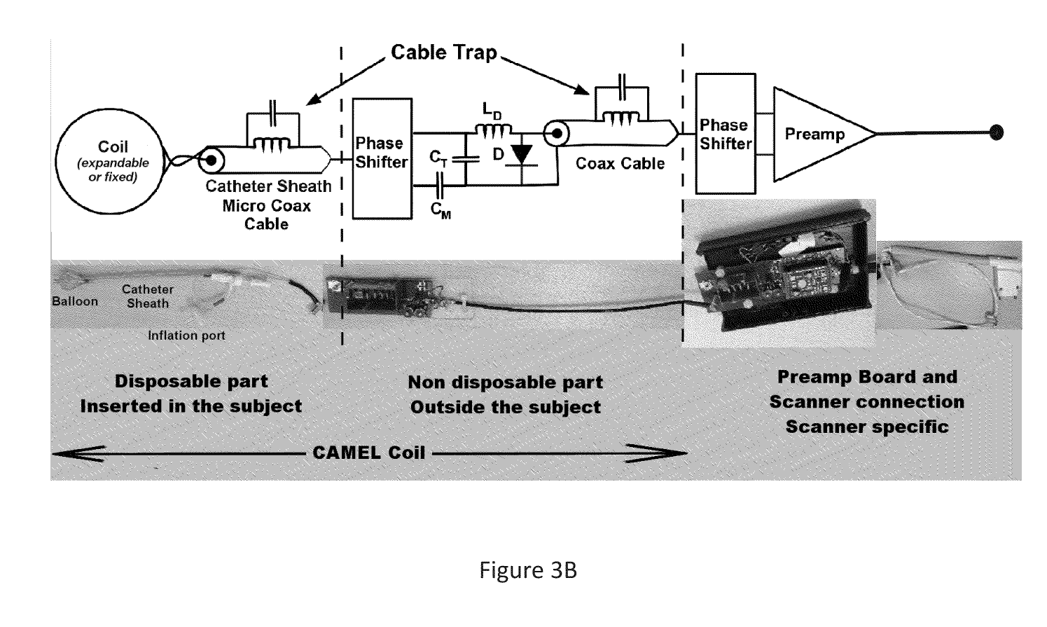

In the embodiments discussed above, the ends of the inductive loop are attached (e.g. using soldering) to a micro-coaxial cable (Axon Cable SAS, Montmirail, France), at a location 2 cm down the sheath and down the filaments or the bottom of the balloon (when expansion mechanism is used). Everything was then sealed within a 10 Fr (10 French=3.3 mm diameter) sheath to protect the electrical connections from moisture and to facilitate connection of the loop to the circuit board at the end of the sheath (FIG. 3A). No passive circuit component was placed in the loop or on the micro-coaxial cable, not only due to space constraints of the small diameter sheath used to mount both coil loops but also to avoid loop improper folding in the case of expandable coils and image phase distortions near the coils. The matching (C_{M}), tuning (C_{T}), and decoupling (L_{D}) circuit elements were placed remote to the inductive loop outside the catheter sheath. For the system shown in FIGS. 1 and 2 used with a 3 T magnet, the circuit values are: C_{T}=10-15 pF; C_{M}=30-50 pF; and L_{D}=7-10 nH. For a different magnet strength and a different coil type, these values will have to be adapted as is known to those skilled in the art.

FIG. 3B shows a photo montage of a CAMEL coil adjacent to the circuit diagram of FIG. 3A indicating which portions are disposable and inserted into the subject (the coil loop and micro-coaxial cable), which are non-disposable and outside of the subject (the phase shifter, circuitry, and coaxial cable), and which are scanner-specific electronics components (preamplifier board and scanner connection).

The micro-coaxial cable connecting the RF coil to the circuit board was 24-cm long (˜λ/10) for phantom testing. It had to be extended to 38.5 cm in order for the coil to access the right atria and ventricle from the jugular access in vivo. A 24-cm (˜λ/10) coaxial cable connected the receiver circuit to the preamplifier board, which included a cable trap to minimize common-mode currents on the cable, a phase shifter to maintain λ/2 phase shift between the coil and a low input impedance preamplifier. Cable traps were used to minimize common mode currents on both coaxial cables. Furthermore, phase shifters ensure that each coax cable results in a λ/2 phase shift between the coil and the preamp.

The coil developed in Volland et al. (Volland N A, Kholmovski E G, Parker D L, Hadley J R. Initial feasibility testing of limited FOV MR thermometry using a local cardiac RF coil. Magnetic Resonance in Medicine 2013, 70 (4): 994-1004, incorporated herein by reference in its entirety) was also constructed with the same 38 AWG copper wire than the two CAMEL coils for comparison. Its tuning and matching circuits were made local to access the losses remote tuning and matching induced in the CAMEL coils. This third coil is referred as to the local coil.

Catheter-Mounted Coil Evaluation

After design and construction, the coil prototype initial evaluations were performed in phantoms to determine the maneuverability and robustness of the coil, its 3D volume sensitivity profiles, and associated SNR maps. Different phantoms were subsequently used to perform magnitude and phase imaging and assess the coil stability and limitations in a moving phantom prior to in vivo use. All the MRI scans were done on either a TIM Trio or a Verio 3 Tesla MRI scanner (Siemens Healthcare, Erlangen, Germany). In various embodiments, the disclosed invention may be used with other MR scanners and magnetic field strengths with minor adaptations.

All the coil characteristics, resonance frequency, quality factor, decoupling factor, were determined unloaded in air and loaded in both static and dynamic saline phantoms. Mechanical stability and consistency of this circuitry were also tested after several cycles of folding and expansion prior to in vivo testing.

For testing purposes, magnitude images were acquired with an SNR-specific Gradient Echo (GRE) pulse sequence with TR=9.1 ms, TE=6 ms, pixel size 1×1 mm², 3-mm slice, flip angle=15°. The FOV was 192×192 mm² in a saline phantom to evaluate the performance and sensitivity gains achieved for all local coils in situ. SNR maps and corresponding profiles were generated for the local coil and compared to the SNR measured for the external coils currently used in the developmental myocardial ablation procedures. The 6-channel torso array was used in combination with a spine coil phased array (Siemens Healthcare, Erlangen, Germany). In various embodiments including clinical uses, other possible imaging sequences include high resolution anatomic imaging, DTI, measuring temperature during thermal ablation treatment, determining local tissue perfusion, or performing MRS for detecting metabolites, as discussed above.

Phantom Imaging Studies

A standard 3D GRE sequence was used to acquire high-resolution magnitude and phase limited FOV image of a kiwi fruit as an example of high-resolution imaging using the disclosed coil. The sequence parameters were the following: TR/TE/flip angle=13.6 ms/6 ms/30°, pixel size=0.4×0.4×0.4 mm³, and bandwidth=200 Hz/pixel. The limited FOV acquired was 96×96 mm².

In Vivo Studies

The feasibility studies will be done in vivo using dog models. All animal experiments will be performed with approval from the local Institution Animal Care and Use Committee (IACUC). The studies will be conducted on yucatan pigs (n=3) as the pig anatomy is comparable to humans. Preparation and MR measurements will be completed under general anesthesia induced by intravenous injection of ketamine and xylazine and maintained by inhalation of isoflurane (2% in oxygen). Surgical cutdowns will be performed to allow intravenous access for the coils through the jugular vein. The animal vital signs will be monitored continuously during the experiments with a Veris MR vital signs monitor (Medrad Inc., Warendale, Pa.). Blood pressure will be kept stable in the range of 160-120/50-80 mmHg at heart rates between 70 and 80 beats per minute. The body internal temperature will be kept at approximately 36° C. A ventilator will maintain the animal respiration rate between 10 and 15 breaths/min. All the MRI acquisitions will be cardiac gated and breath hold will be applied. The phase encode direction will be set perpendicular to the coil axis to minimize motion artifacts.

A 12fr 30 cm long sheath will be used to allow the coil to travel the vasculature before expanding in the heart chamber (FIG. 1b). This sheath will orient the coil to enable adequate MR imaging in the heart chambers in regards to B₀. The local coil will be originally folded in the sheath. Extra coaxial cables of 24-cm length (RG-316, Thermal wire and cable, LLC, Naples, Fla.) with cable traps may be added to the coil circuit to keep the preamplifier circuit away from the animal. For MR measurements, the animal will be placed supine head first on the MR patient table.

Image acquisition will be performed as during the phantom studies and cardiac gating will be optimized to minimize motion artifacts in the images. A cine imaging sequence will be performed using the external coil arrays to find the stationary phase of cardiac cycle in the ROI, and the corresponding value for the post-QRS delay. Intermittent breath-hold, achieved by turning the ventilator off, will be used to minimize motion artifacts in the images. The cine imaging sequence will also be used to quantify the coil motion and deformation in vivo.

Catheter-Mounted Coil Characteristics

All the coils were tuned at 123.23 MHz (3 T); however, depending on the particular system that is used, the coils may be tuned to 1.5 T or any magnet strength desired. The maximum return loss was −20 dB when the coils were loaded upon repeated folds and expansions. The coils achieve a maximum active detuning of −20 dB to properly decouple the local coils during RF excitation. The quality factor (Q) of the different coils loaded and unloaded is summarized in Table 1. The Q standard deviation represents the value variation after at least 5 folds and expansions. The resonant frequency was varying no more than 0.5 MHz upon the successive folds and expansions for all the coils tested and did not evolved retuning the coil before magnet testing.

TABLE 1
CAMEL Coil prototype performances after repeated cycles of
folds and expansions (n ≧ 5) vs. local coil performances.

| | | | | | Balloon Coil | | | |
|---|---|---|---|---|---|---|---|---|
| | | | Umbrella Coil | | Air Inflated | | Fluid Inflated | |
| | Local coil | | (b) | | (c) | | (d) | |
| | (a) | | Expanded | | | | | |
| | Q_{unloaded} | Q_{loaded} | Q_{unloaded} | Q_{loaded} | Q_{unloaded} | Q_{loaded} | Q_{unloaded} | Q_{loaded} |
| Average | 31.6 | 21.8 | 32.2 | 26.4 | 38.8 | 34.8 | 37.6 | 39 |
| Standard | 1.5 | 0.8 | 1.9 | 0.3 | 0.7 | 0.9 | 1 | 0.7 |
| Deviation | | | | | | | | |
| Q_{U}/Q_{L} | | 1.5 | | 1.2 | | 1.1 | | 1 |

Sensitivity Profile and SNR Studies:

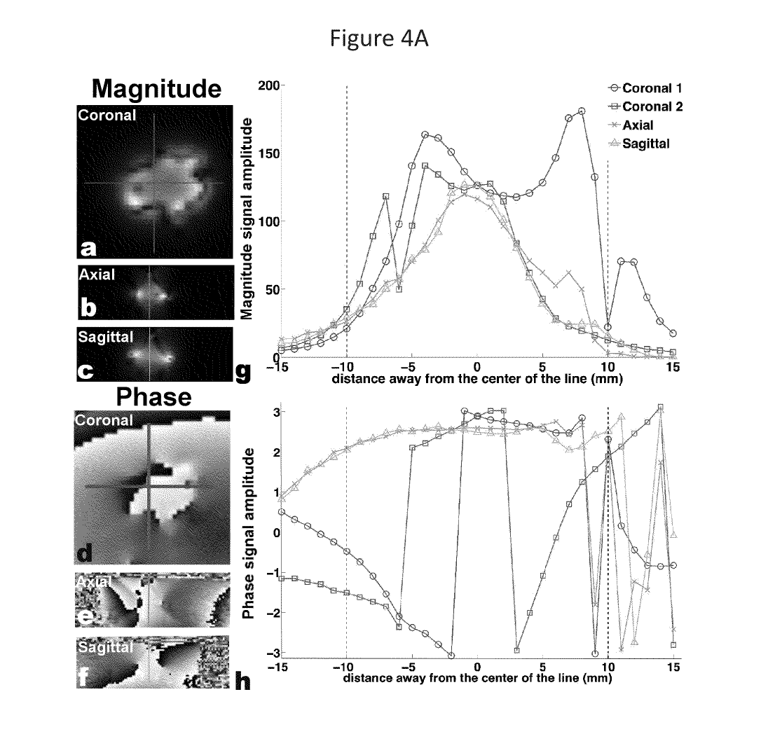

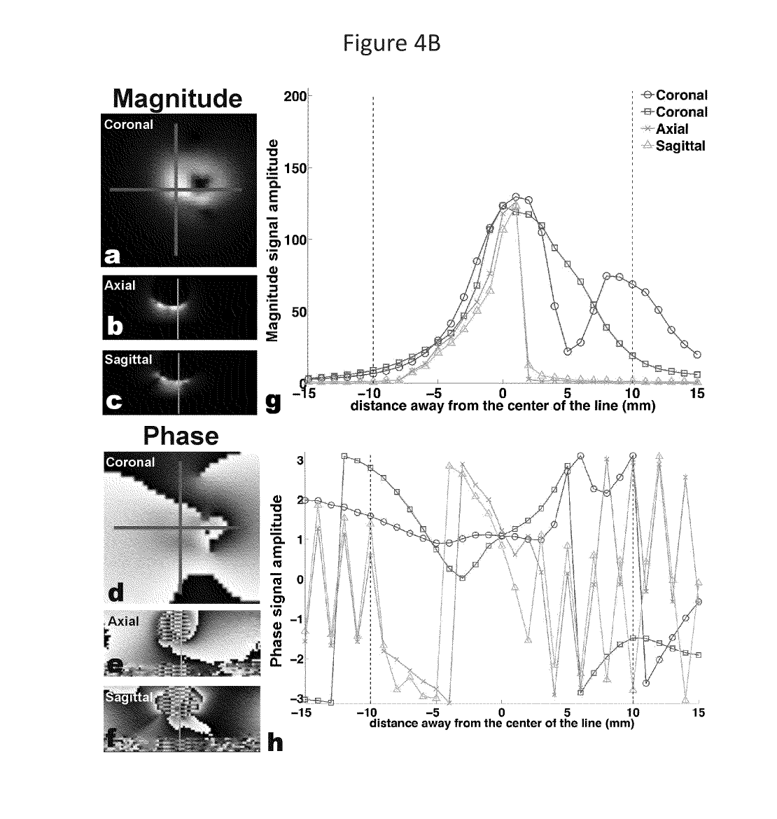

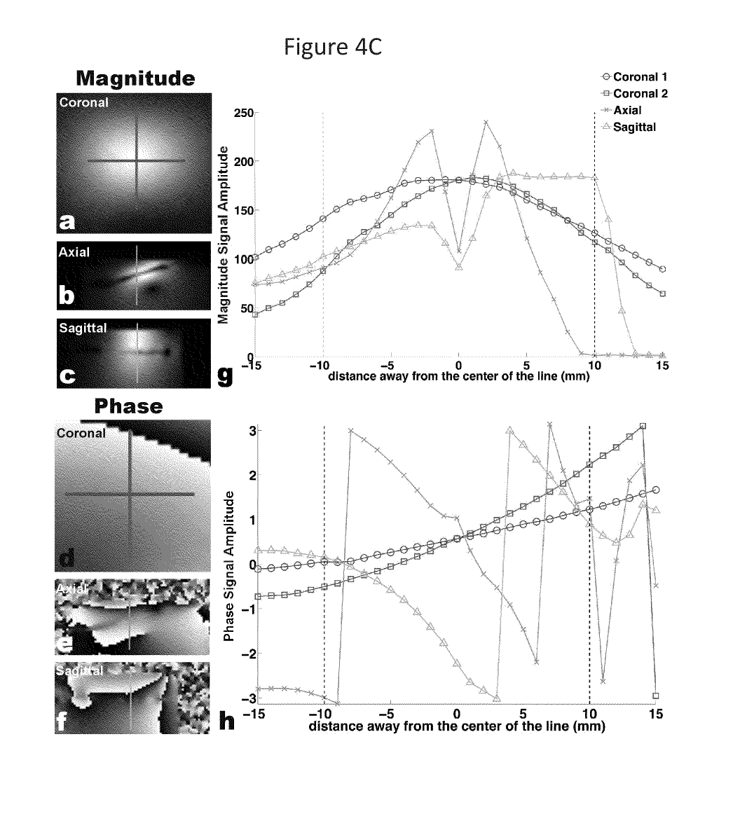

The SNR images acquired in the three directions of space in a saline phantom using the two different types of CAMEL coils are shown in FIGS. 4A and 4B and the local coil in FIG. 4C. FIG. 4A presents the magnitude and phase images acquired in three different orientations (coronal, axial, and sagittal to the coil) using the umbrella coil. FIG. 4B presents the magnitude and phase images acquired in three different orientations (coronal, sagittal, and axial to the coil) using the balloon coil. FIG. 4C presents the magnitude and phase images acquired in three different orientations (coronal, sagittal, and axial to the coil) using the local coil. The coil sensitivities were slightly different between the balloon coil and the umbrella and local coils. The balloon coil only detected signal in front of the balloon because of the fluid contained within the balloon. The signal void in the middle of the coil was due to the catheter sheath going through to allow the balloon deployment and RF ablation catheter positioning. The sensitivity of coils such as the 3 described here, with an SNR is higher than 500, extend 0.5 (balloon) to 1 cm in front of the coil for the diameter of the coil. Very weak signal sensitivity was detected less than 1 cm away from the coils. The SNR was 1175 (±17%) on average for the local coil, 657 (±11%) for the umbrella coil, 539 (±9%) for the balloon coil within the sensitivity volume of the coils (FIGS. 4A, 4B, 4C). This is over 50 times higher than what the external coil arrays would allow at the depth of the heart in vivo (12±15%). The phase images show smoothly varying signals across the sensitivity volume (FIGS. 4A, 4B, 4C).

High Resolution Imaging Studies

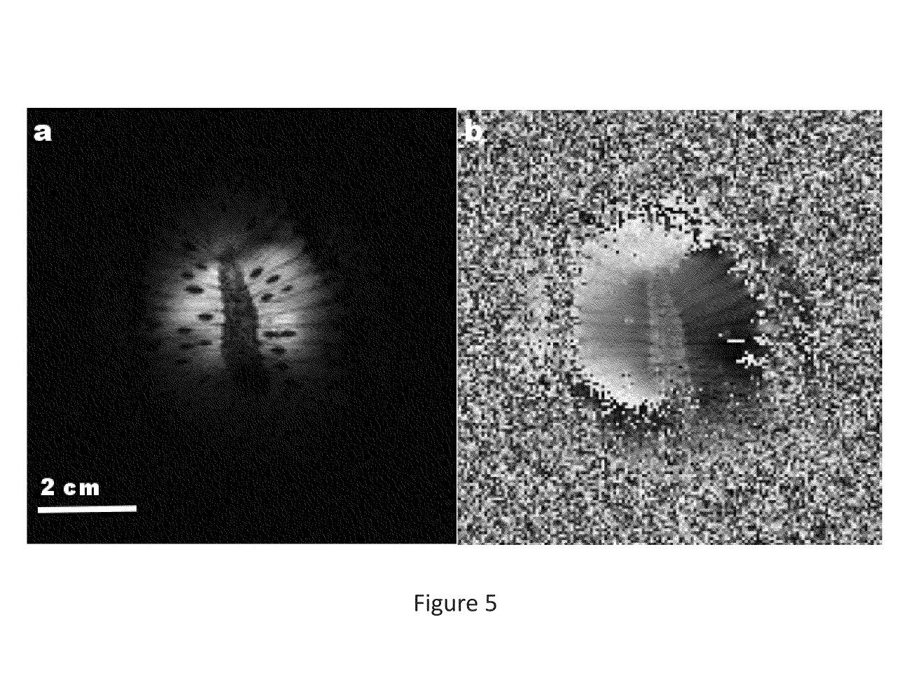

3D high-resolution (0.4×0.4×0.4 mm³) images of a kiwi fruit are presented in FIG. 5. These images were acquired in 139 sec. The signal sensitivity volume of the coils allowed seeing very fine details of the fruit close to the coil on the magnitude images but not outside the coil. The phase images have a smooth signal variation across the sensitivity volume of the coil as well. The phase signal varies by less than 15% from pixel to pixel across the sensitivity region.

Accordingly, disclosed herein is the development of two types of CAMEL coils to allow rapid image acquisition within the heart with high resolution and reduced motion artifacts. It is demonstrated that such a coil can travel the vasculature and expand and fold in the heart to allow acquisition of images of the myocardium surface. The image quality obtained using such coils is adequate to be clinically useful to get information never accessible before, thereby increasing diagnostic capability. The substantial advantages of the CAMEL coil over external and local coils are the minimal invasiveness, the relative ease of use, and the rapid insertion into the vasculature. The advantages make them well suited for further development toward clinical use.

Although Nitinol is of interest as a coil substrate because of its shape memory properties and wide use in foldable/expandable medical devices, such as the Lasso mapping catheter (BiosenseWebster, Diamond Bar, Calif.) and the constellation full contact mapping catheter (Boston Scientific, Natick, Mass.), the susceptibility artifacts from its nickel content (close to 50%) causes signal loss and distortions in the limited FOV MR images, rendering them unacceptable for analysis since the regions of interest (ROI) were within a couple of millimeters of the coil wire. Furthermore, when considering MR thermometry, TE has to be long enough to give accurate temperature measurement (>6 ms) creating even larger image artifact especially at 3 T. For these studies, copper was used as the coil substrate because of its high conductivity and common used in MR coil manufacturing. However, a biocompatible substrate such as silver or gold may be used for clinical use. The disclosed catheter-mounted coil may be used with MR scanners having other field strengths, including 1.5 T, with suitable adaptations being made to the apparatus and procedures including an echo time TE of at least about 10 ms for obtaining phase images, e.g. as used in MR thermometry studies. In various embodiments and with different magnetic field strengths, TE is at least 5 ms, at least 6 ms, at least 7 ms, at least 8 ms, at least 9 ms, at least 10 ms, at least 11 ms, at least 12 ms, at least 13 ms, at least 14, or at least 15 ms.

The umbrella design (FIG. 1) has some advantages over the balloon design. First of all, with this design the blood flow is minimally restricted compared to the balloon design (FIG. 2). Such a coil is also very easy to push in and out of the catheter sheath to be used. However, the balloon design was more mechanically stable in the blood flow than the umbrella design despite its larger reduction of the blood flow. It was also slightly more complex to expand and fold the coil as air or fluid had to be injected or aspirated and the balloon had to be shaped upon folding and expansion. Furthermore, the damage on the filament arms could have more dramatic consequences for the patient than a hole in the balloon filled with biocompatible fluid, such as perfluorocarbons or zero-susceptibility liquid, to decrease artifact in the images.

Regardless of which expansion mechanism is used, if any, the coil when in use needs to maximize forward tissue detection and for expandable coils the configuration of the coil needs to be consistent and repeatable upon deployment. In some embodiments the inductive coil loop is placed into a circular configuration (either in a fixed position or after expansion) during use. In other embodiments the coil inductive loop may have other configurations such as oval, ellipsoidal, butterfly or multi-lobed shaped. Smaller coils (e.g. 2 cm diameter or less) have an advantage of facilitating higher time resolution (e.g. 70 ms per image or less), although the working distance (depth of penetration) of such coils is relatively short (e.g. about 1 cm for a circular coil with a 2 cm diameter). In general, the coil is arranged so that in its working configuration its inductive loop is disposed in a plane which is perpendicular to the long axis of the catheter sheath to which the coil is attached, although in various embodiments the coil is disposed in planes in various other orientations relative to the catheter sheath. In some embodiments, the coil has a shape that is not planar and may, for example, be curved rather than planar.

Due to size constraints, the tuning, matching, and decoupling circuits were set remotely (FIG. 3). This does affect the performances of the CAMEL coils compared to the local coil. However, with the high SNR available for such coil (FIG. 4), a 44% loss is not of importance compared to the gain in information that the high resolution images obtained can give compared to external coil (still 25 times higher sensitivity).

When the balloon was filled with air, large image artifacts were present in both the magnitude and phase images. This was due to the air/liquid interface. It largely impacted the performance of the coil and made it unsafe and unacceptable to use in a clinical setting. It was only used during early stages of development for inflation and leak detection. For all future experiments, MnCl₂ saline solution will be used.

The 21% SNR difference between the umbrella and balloon coil images could be due to the difference in copper wire used (pure copper versus copper plated silver) and coating materials (Balloon thin Polyurethane film versus Teflon) used to build the 2 coils. The balloon-type coil also has less homogeneity throughout its sensitivity volume because of the small (<1 mm) and not irrigated lumen present at the distal end of the balloon (FIG. 4). This lumen is designed to allow the insertion of a catheter, such as an ablation catheter, that might be needed during a catheter-based MR-guided intervention. Such insertion would require irrigation and should reduce the inhomogeneity of the coil sensitivity volume.

In various embodiments, longer coaxial cables may be used to connect the CAMEL coil, as the standard point of catheter insertion is the femoral artery.

In order to allow imaging acquisition in the entire heart regardless of the coil orientation and the sheath used, the coil inductive loop design and flexibility will, in various embodiments, be adapted to compensate for B₀ insensitivity when the coil axis is parallel to the main magnetic field.

The high SNR in the small sensitivity volume of the coil is an essential advantage to perform high-resolution anatomic imaging, DTI, determining local tissue perfusion, or performing MRS. The smooth phase transition in such a localized volume is another advantage to perform temperature measurements and obtain accurate results. Furthermore, high-resolution images will improve the temperature measurement accuracy.

This work has shown the development of two types of CAMEL coils to enable the acquisition of rapid, stable, and accurate high-resolution magnitude and phase images from the cardiac wall of a beating heart with minimal invasiveness. The sensitivity improvement obtained using the two types of CAMEL coils allows the use of the coils for high resolution and fast imaging which could be applied to real-time monitoring and visualization of lesion formation during RF ablation procedures as an example. This capability may allow for the characterization of tissue damage and aid with the prediction of the outcome effectiveness following a catheter-based procedure for example.

Although the disclosure above is given in terms of a coil for use with a catheter in cardiac environments, in other embodiments the coil may be used with catheters for probing other tissues including the kidney, liver (with microsolenoid coils), colon, prostate, rectum, cervix (larger catheter-mounted coils), uterus, bladder, and stomach (expandable coils). In various embodiments the coil may be used as a receive coil or as a transceiver coil.

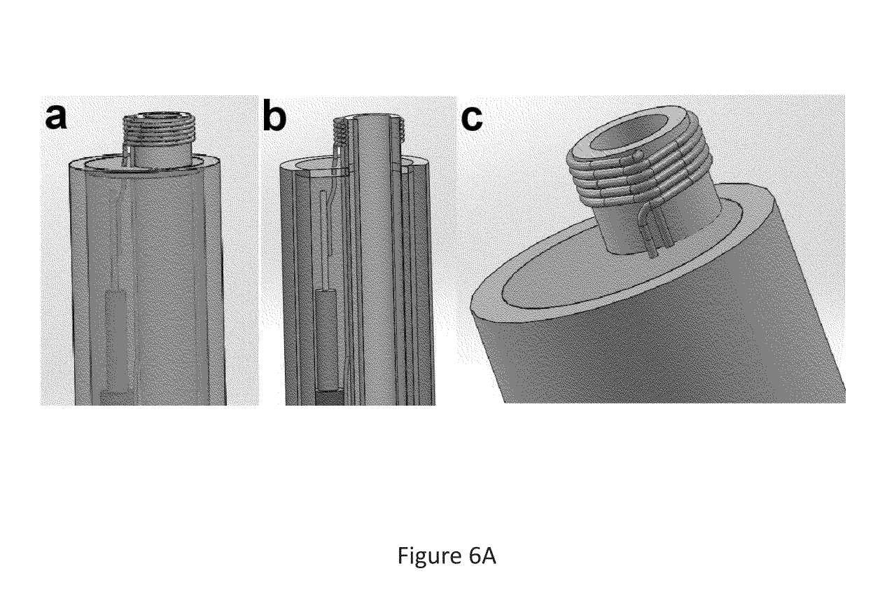

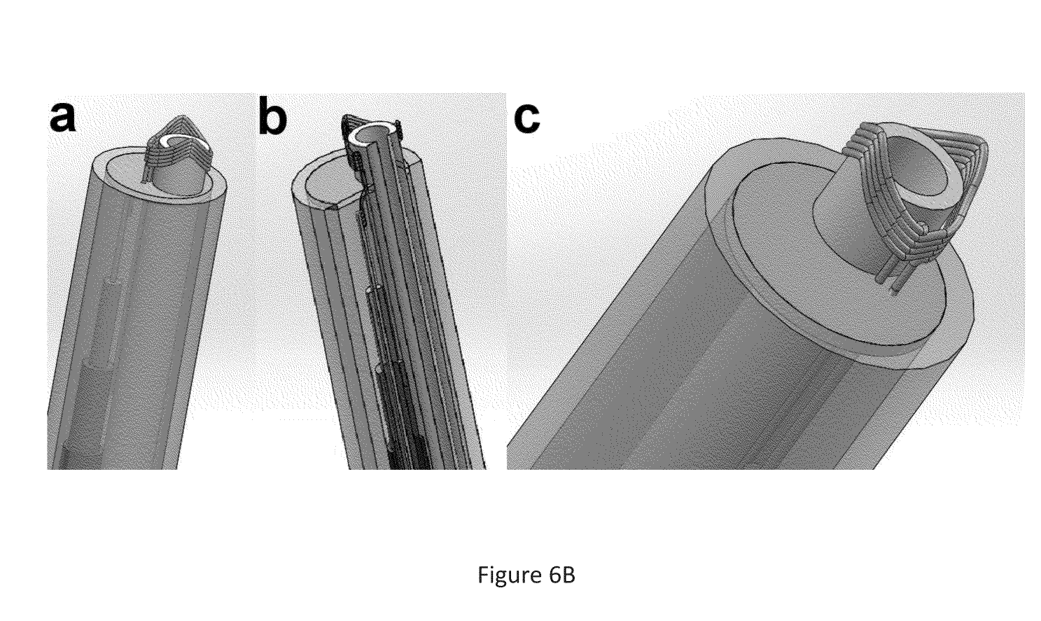

In one particular embodiment, a coil as disclosed herein may be used with an ablation catheter during a catheter-based radiofrequency renal artery sympathetic denervation procedure. This procedure involves locating a catheter with an ablation electrode extending from the end in a patient's renal artery to denervate near the vascular wall in order to treat the patient's hypertension. Given the relatively small size of the renal artery (approx. 5 mm inner diameter), in various embodiments a catheter-mounted coil for applications such as this may be fixed, rather than expandable, as the maximum coil size is comparable to the cross-sectional size of the catheter sheath (FIG. 6A). In some embodiments, the coil includes a microsolenoid inductive loop positioned near or at the end of the catheter sheath (FIG. 6B). During or immediately following RF ablation (e.g. of the sympathetic nerves along the wall of the renal artery) the coil may be used to obtain images (which in some embodiments may include MR thermometry images) of the adjacent tissue to monitor and assess the progress of ablation.

Various features and advantages of the invention are set forth in the following claims.

What is claimed is:

1. An expandable catheter-mounted coil for magnetic resonance imaging, comprising:
a catheter sheath comprising an elongated tube with a central axis, the catheter sheath having an opening at an end thereof;
an expandable coil comprising a conductive material connected to an expansion mechanism which, when deployed, maintains the expandable coil shape; and
a cable running through the catheter sheath, the cable being electrically connected to the expandable coil.

2. The expandable catheter-mounted receive coil of claim 1, wherein the shape of the expandable coil is planar and wherein the plane of the expandable coil is perpendicular to the central axis of the catheter sheath.

3. The expandable catheter-mounted receive coil of claim 1, wherein the expansion mechanism comprises a first balloon, wherein the expandable coil is maintained in a position adjacent a perimeter of the first balloon, and wherein inflation of the first balloon maintains the expandable coil shape.

4. The expandable catheter-mounted receive coil of claim 3, wherein the first balloon is inflated with saline solution comprising MnCl₂.

5. The expandable catheter-mounted receive coil of claim 3, wherein the expandable receive coil is attached at a plurality of discrete locations to the perimeter of the first balloon.

6. The expandable catheter-mounted coil of claim 3, wherein expansion mechanism further comprises a second balloon and wherein the expandable receive coil is disposed between the first balloon and the second balloon.

7. The expandable catheter-mounted receive coil of claim 1, wherein the expansion mechanism comprises a plurality of resilient filaments slidingly disposed within the catheter sheath and biased away from the central axis, wherein the expandable coil is attached at an end of each filament, and wherein sliding the filaments out of the catheter sheath allows the filaments to move away from the central axis and maintain the expandable receive coil shape.

8. The expandable catheter-mounted coil of claim 1, wherein the cable comprises a coaxial cable.

9. The expandable catheter-mounted coil of claim 8, further comprising a phase shifter electrically connected to the cable.

10. The expandable catheter-mounted coil of claim 9, wherein the cable comprises a circuit electrically connected to the phase shifter, the cable, and the expandable coil, the circuit configured to perform at least one of matching, tuning, and decoupling.

11. The expandable catheter-mounted coil of claim 10, wherein the expandable coil is configured for use within a 3 Tesla MR coil.

12. The expandable catheter-mounted coil of claim 1, wherein the expandable inductive loop coil comprises gold or silver.

13. A method for performing magnetic resonance imaging, the method comprising the steps of:
providing a catheter sheath comprising an elongated tube with a central axis, the catheter sheath having an opening at an end thereof;
disposing a cable within the catheter sheath;
electrically connecting an expandable coil to the cable, the expandable coil comprising a conductive material connected to an expansion mechanism;
deploying the expansion mechanism to maintain the expandable coil shape; and
placing the expandable coil within a magnetic field to obtain data.

14. The method of claim 13, wherein the shape of expandable coil is planar and wherein the plane of the expandable coil is perpendicular to the central axis of the catheter sheath.

15. The method of claim 13, wherein the expansion mechanism comprises a first balloon, the method further comprising
maintaining the expandable coil in a position adjacent a perimeter of the first balloon, and inflating the first balloon to maintain the expandable coil shape.

16. The method of claim 15, further comprising inflating the first balloon with saline comprising MnCl₂.

17. The method of claim 15, further comprising attaching the expandable coil at a plurality of discrete locations to the perimeter of the first balloon.

18. The method of claim 15, wherein expansion mechanism further comprises a second balloon and wherein the expandable coil is disposed between the first balloon and the second balloon.

19. The method of claim 13, wherein the expansion mechanism comprises a plurality of resilient filaments slidingly disposed within the catheter sheath and biased away from the central axis, the method further comprising
attaching the expandable coil at an end of each filament, and
sliding the filaments out of the catheter sheath to allow the filaments to move away from the central axis and maintain the expandable receive coil shape.

20. The method of claim 13, wherein the cable comprises a coaxial cable.

21. The method of claim 20, further comprising electrically connecting a phase shifter to the cable.

22. The method of claim 21, further comprising electrically connecting a circuit to the phase shifter, the cable, and the expandable receive coil, the circuit configured to perform at least one of matching, tuning, and decoupling.

23. The method of claim 22, wherein the magnetic field is generated by a commercial MR scanner.

24. The method of claim 23, further comprising applying a gradient echo pulse sequence having an echo time of at least 6 ms.

25. The method of claim 24, further comprising obtaining a phase image.

26. The method of claim 25, further comprising performing MR thermometry using the phase image.

27. The method of claim 13, wherein the expandable coil comprises silver or gold.

28. A method for performing magnetic resonance imaging, the method comprising the steps of:
providing a catheter sheath comprising an elongated tube with a central axis, the catheter sheath having an opening at an end thereof;
disposing a cable within the catheter sheath;
electrically connecting a coil to the cable, the coil comprising a conductive material;
maintaining the coil shape; and
locating the catheter sheath with the coil within a magnetic field to obtain data.

29. The method of claim 28, further comprising applying a gradient echo pulse sequence having an echo time of at least 6 ms.

30. The method of claim 29, further comprising obtaining a phase image.

31. The method of claim 30, further comprising performing MR thermometry using the phase image.

32. The method of claim 28, wherein the cable comprises a coaxial cable.

33. The method of claim 32, further comprising electrically connecting a phase shifter to the cable.

34. The method of claim 33, further comprising electrically connecting a circuit to the phase shifter, the cable, and the coil, the circuit configured to perform at least one of matching, tuning, and decoupling.

35. The method of claim 28, wherein locating the catheter sheath with the coil within a magnetic field to obtain data further comprises locating the catheter sheath within a renal artery of a patient.
